(19) United States
(12) Patent Application Publication
(10) Pub. No.: US 2015/0265222 A1
(43) Pub. Date: Sep. 24, 2015

(54) MEDICAL INFORMATION PROCESSING APPARATUS, MEDICAL IMAGE DIAGNOSTIC APPARATUS, AND MEDICAL INFORMATION PROCESSING METHOD

(71) Applicants: Kabushiki Kaisha Toshiba, Minato-ku (JP); Toshiba Medical Systems Corporation, Otawara-shi (JP)

(72) Inventor: Takuya SAKAGUCHI, Utsunomiya (JP)

(73) Assignees: Kabushiki Kaisha Toshiba, Minato-ku (JP); Toshiba Medical Systems Corporation, Otawara-shi (JP)

(21) Appl. No.: 14/723,959

(22) Filed: May 28, 2015

(30) Foreign Application Priority Data
Nov. 29, 2012 (JP) 2012-260948
Nov. 27, 2013 (JP) 2013-245391

(51) Int. Cl. A61B 5/00; A61B 8/08; A61B 6/03; A61B 5/026; A61B 5/02

(52) U.S. Cl. CPC A61B 5/7282; A61B 5/0263; A61B 5/02007; A61B 5/743; A61B 6/032; A61B 6/037; A61B 8/0891; A61B 2576/00

(57) ABSTRACT

A medical information processing apparatus according to embodiments includes processing circuitry. The processing circuitry configured to generate display information indicating a state of a first region in a tissue of a subject and a state of a second region in a feeding vessel of the first region, depending on flow reserve of the first region and fractional flow reserve of the second region. The processing circuitry configured to execute control such that the display information generated is shown by a display unit.

### CROSS-REFERENCE TO RELATED APPLICATIONS

This application is a continuation-in-part (CIP) of PCT international application Ser. No. PCT/JP2013/081977 filed on Nov. 27, 2013 which designates the United States, incorporated herein by reference, and which claims the benefit of priority from Japanese Patent Application No. 2012-260948, filed on Nov. 29, 2012 and Japanese Patent Application No. 2013-245391, filed on Nov. 27, 2013, the entire contents of which are incorporated herein by reference.

### FIELD

Embodiments described herein relate generally to a medical information processing apparatus, a medical image diagnostic apparatus, and a medical information processing method.

### BACKGROUND

Conventionally, as a useful diagnostic index for diagnosing ischemic cardiac disease that is developed by insufficient blood flow due to a coronary stenosis, coronary flow reserve (CFR) and fractional flow reserve (FFR) have been known. The CFR is a ratio of blood flow of a coronary artery at rest (a coronary blood flow at rest) to blood flow of the coronary artery at peak hyperemia at which blood vessels are dilated to a maximum degree (a coronary blood flow at peak hyperemia), and is an index indicating the degree of ischemia. In other words, the CFR is an index indicating an ability that can increase the coronary blood flow.

The FFR is a rate of blood flow at peak hyperemia when there is a stenosis in a coronary artery, assuming that the blood flow at peak hyperemia when there is no stenosis in the coronary artery is “1.0”, and is an index indicating the degree of the stenosis. In other words, the FFR is an index indicating a percentage of coronary blood flow with respect to the coronary blood flow at normal. Generally, the FFR is calculated by a ratio of a peripheral coronary artery pressure to an aortic coronary artery pressure having a stenosis site put therebetween.

In recent years, at the time of a diagnosis of ischemic cardiac disease and a decision of a treatment method thereof, complex usage of the indexes mentioned above such as CFR and FFR has been desired. For example, when it is judged whether to perform PCI (Percutaneous Coronary Intervention), the decision of the treatment method under such conditions that ischemia is present in the cardiac muscle and it is caused by a stenosis can be considered. In this case, a doctor selects the PCI as the treatment method when the CFR is low (for example, CFR<2) and the FFR is low (for example, FFR<0.8). However, in the conventional techniques described above, complex usage of the indexes may not be performed easily.

### BRIEF DESCRIPTION OF THE DRAWINGS

FIG. 1 is an example of a configuration of a medical information processing system according to a first embodiment;

FIG. 2 is an explanatory diagram of a first example of complex usage of a plurality of indexes according to the first embodiment;

FIG. 3A is an explanatory diagram of calculation of CFR according to the first embodiment;

FIG. 3B is another explanatory diagram of calculation of CFR according to the first embodiment;

FIG. 4A is an explanatory diagram of FFR calculation according to the first embodiment;

FIG. 4B is another explanatory diagram of FFR calculation according to the first embodiment;

FIG. 5 is an example of a configuration of a medical information processing apparatus according to the first embodiment;

FIG. 6 are examples of generation of display information by a generation unit according to the first embodiment;

FIG. 7 is an example of information displayed under control of a display control unit according to the first embodiment;

FIG. 8 is an explanatory diagram of a second example of complex usage of a plurality of indexes according to the first embodiment;

FIG. 9 are examples of generation of display information by the generation unit according to the first embodiment;

FIG. 10 are examples of a graph to be generated by the generation unit according to the first embodiment;

FIG. 11 are examples of display information to be display-controlled by the display control unit according to the first embodiment;

FIG. 12 are examples of a change of display information, which follows a region change by the medical information processing apparatus according to the first embodiment;

FIG. 13A is a display example of display information according to the first embodiment;

FIG. 13B is another display example of display information according to the first embodiment;

FIG. 14A is an example of display information generated by the medical information processing apparatus according to the first embodiment;

FIG. 14B is another example of display information generated by the medical information processing apparatus according to the first embodiment;

FIG. 15 is a flowchart of a process procedure performed by the medical information processing apparatus according to the first embodiment;

FIG. 16 is an example of display information generated by a generation unit according to a second embodiment;

FIG. 17 are examples of information displayed under control of a display control unit according to the second embodiment;

FIG. 18 is an example of display information to be displayed by a medical information processing apparatus according to a third embodiment;

FIG. 19 is another example of display information to be displayed by the medical information processing apparatus according to the third embodiment;

FIG. 20A is an example of display information to be displayed by the medical information processing apparatus according to the third embodiment; and

FIG. 20B is another example of display information to be displayed by the medical information processing apparatus according to the third embodiment.

FIG. 21 is a diagram illustrating an example of the configuration of a medical information processing apparatus according to another embodiment.

### DETAILED DESCRIPTION

According to an embodiment, a medical information processing apparatus includes processing circuitry. The processing circuitry configured to generate display information indicating a state of a first region in a tissue of a subject and a state of a second region in a feeding vessel of the first region, depending on flow reserve of the first region and fractional flow reserve of the second region. The processing circuitry execute control such that the display information generated by the generation unit is shown by a display.

### First Embodiment

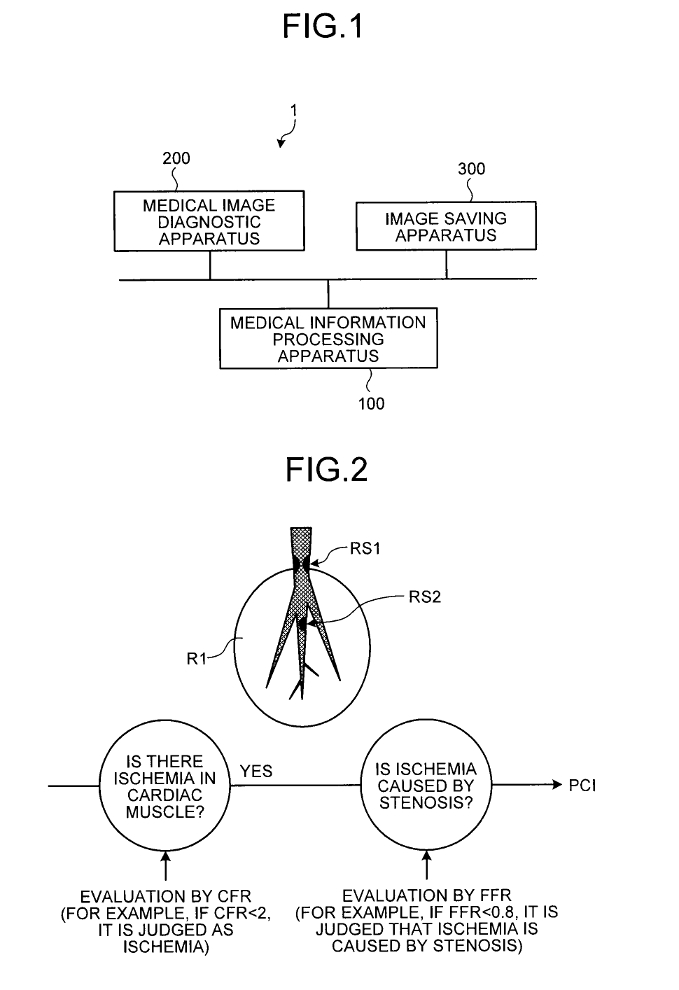

A medical information processing apparatus according to the present application is explained below in detail. In a first embodiment, a medical information processing system including a medical information processing apparatus according to the present application is explained as an example. FIG. 1 is an example of a configuration of a medical information processing system 1 according to the first embodiment.

As shown in FIG. 1, the medical information processing system 1 according to the first embodiment includes a medical information processing apparatus 100, a medical image diagnostic apparatus 200, and an image saving apparatus 300. Respective apparatus shown in FIG. 1 are in a communicable state with each other directly or indirectly by a hospital LAN (Local Area Network) installed in a hospital. For example, when a PACS (Picture Archiving and Communication System) has been introduced in the medical information processing system 1, the respective apparatus transmit and receive medical images and the like to and from each other based on the DICOM (Digital Imaging and Communications in Medicine) standard.

The medical image diagnostic apparatus 200 is, for example, an X-ray diagnostic apparatus, an X-ray CT (Computed Tomography) scanner, an MRI (Magnetic Resonance Imaging) scanner, an ultrasonograph, an SPECT (Single Photon Emission Computed Tomography) scanner, a PET (Positron Emission Computed Tomography) scanner, an SPECT-CT scanner in which the SPECT scanner and the X-ray CT scanner are integrated, a PET-CT scanner in which the PET scanner and the X-ray CT scanner are integrated, or these scanner groups. The medical image diagnostic apparatus 200 collects medical images according to operations performed by each technician.

Specifically, the medical image diagnostic apparatus 200 collects image data of various images associated with a diagnosis and treatment of ischemic cardiac disease. For example, the medical image diagnostic apparatus 200 collects image data of medical images for measuring the coronary flow reserve (CFR) and the fractional flow reserve (FFR), which are diagnostic indexes for diagnosing ischemic cardiac disease, and a stenosis ratio of a stenosis having occurred in a coronary artery. The medical image diagnostic apparatus 200 can calculate respective index values by using the collected image data.

The medical image diagnostic apparatus 200 generates a medical image for measuring the diagnostic indexes described above by a medical apparatus. For example, the X-ray diagnostic apparatus as the medical image diagnostic apparatus 200 generates a perspective image, which is referred to for measurement of the FFR by a pressure wire. That is, a doctor measures the FFR by inserting the pressure wire into a stenosis site, while referring to the perspective image generated by the X-ray diagnostic apparatus.

The medical image diagnostic apparatus 200 transmits the collected image data to the image saving apparatus 300. The medical image diagnostic apparatus 200 transmits, as accompanying information, for example, a patient ID that identifies a patient, a test ID that identifies a test, an apparatus ID that identifies the medical image diagnostic apparatus 200, and a series ID that identifies one shooting by the medical image diagnostic apparatus 200, at the time of transmitting the image data to the image saving apparatus 300. When the medical image diagnostic apparatus 200 calculates the respective index values, the medical image diagnostic apparatus 200 also transmits the calculated values as the accompanying information of the image data.

The image saving apparatus 300 is a database that stores therein medical images. Specifically, the image saving apparatus 300 stores the image data and accompanying information of the respective pieces of image data transmitted from the medical image diagnostic apparatus 200 in a storage unit and saves these pieces of information. The image saving apparatus 300 stores and saves the respective index values measured by using the medical apparatus in the storage unit together with the image used for the measurement.

The medical information processing apparatus 100 acquires the image data from the medical image diagnostic apparatus 200 or the image saving apparatus 300 to generate display information that enables to simplify complex usage of a plurality of indexes, and displays the display information. The complex usage of indexes is explained below. The complex usage of a plurality of indexes means complex usage of diagnostic indexes of the ischemic cardiac disease, such as the CFR and the FFR described above, to perform a diagnosis of the ischemic cardiac disease and a decision of a treatment method thereof. An example of the complex usage of the indexes is explained below.

FIG. 2 is an explanatory diagram of a first example of complex usage of a plurality of indexes according to the first embodiment. In an upper part of FIG. 2, a predetermined region in the cardiac muscle and a coronary artery dominant in the region (a feeding vessel) are shown. As the complex usage of the indexes, for example, as shown in FIG. 2, when there is ischemia in the cardiac muscle and the cause of ischemia is a stenosis, a decision of a treatment method thereof in which the PCI is performed with respect to the stenosis site can be mentioned.

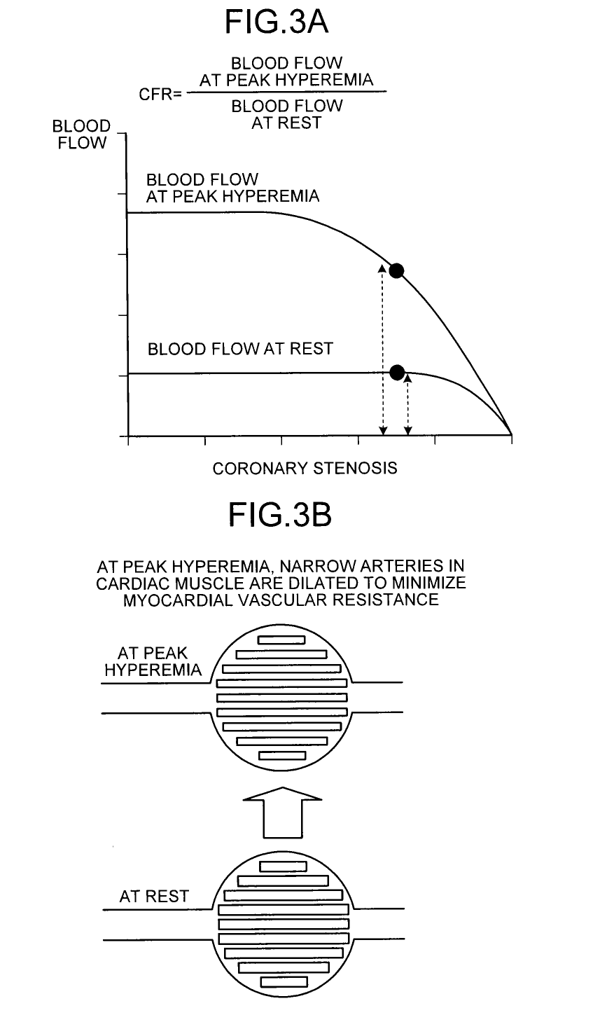

As an example, as shown in FIG. 2, it is determined whether a region R1 in the cardiac muscle has ischemia. Evaluation of whether there is ischemia is performed by using the CFR. As this evaluation, for example, it is determined that there is ischemia if “CFR<2”. The CFR is a useful index indicating the degree of ischemia, and is calculated by “CFR=blood flow at peak hyperemia/blood flow at rest” as shown in FIG. 3A. The blood flow at peak hyperemia here indicates blood flow dilated to the maximum degree, and the blood flow at rest indicates blood flow in a state with the blood vessel not being dilated. That is, as shown in FIG. 3B, at peak hyperemia, narrow arteries in the cardiac muscle are dilated and a myocardial vascular resistance is minimized, and the blood flow increases as compared with the blood flow at rest.

A relation between a blood flow and a coronary stenosis is shown in FIG. 3A. In FIG. 3A, the blood flow is plotted on a y-axis and a stenosis ratio of a coronary stenosis is plotted on an x-axis. As shown in FIG. 3A, the blood flow at peak hyperemia is four to five times the blood flow at rest. The blood flow at rest does not drop even with a stenosis ratio of 80% to 90%, whereas the blood flow at peak hyperemia drops even with a stenosis ratio of about 50%. Therefore, when “CFR=blood flow at peak hyperemia/blood flow at rest” is calculated, a stenosis is present in the coronary artery and the value thereof decreases near a point at which the stenosis ratio exceeds 50%. In the CFR measurement, the degree of ischemia is evaluated by utilizing this characteristic. FIGS. 3A and 3B are explanatory diagrams of calculation of CFR according to the first embodiment.

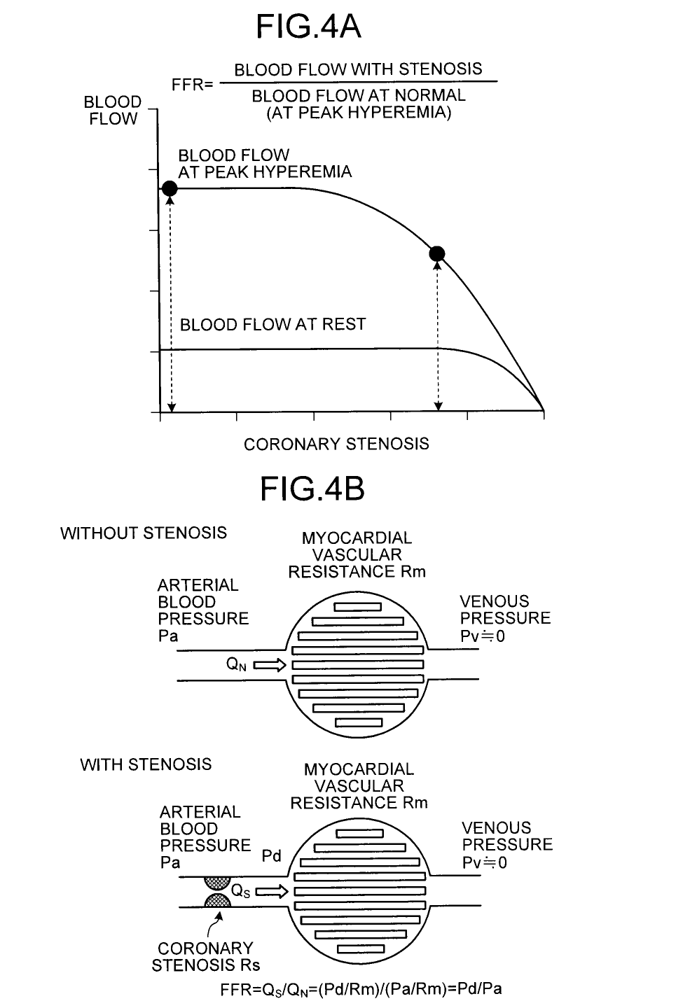

Referring back to FIG. 2, for example, when it is determined that the region R1 has ischemia by the evaluation method described above, it is then determined whether the cause of ischemia is a coronary stenosis RS1 and a coronary stenosis RS2. It is evaluated here whether the coronary stenosis is the cause of ischemia by the FFR in the stenosis. As this evaluation, for example, when “FFR<0.8”, it is determined that the cause of ischemia is the stenosis. The FFR is a useful index indicating the degree of the stenosis, and is calculated, as shown in FIG. 4A, by “FFR=blood flow with stenosis/blood flow at normal”.

In FIG. 4A, a graph similar to that shown in FIG. 3 is shown. As shown in FIG. 4A, the FFR indicates a rate of blood flow at peak hyperemia with a stenosis to blood flow at peak hyperemia at normal (with a stenosis of 0%). That is, in the FFR measurement, the blood flow at peak hyperemia is used at which the blood flow is likely to change depending on a change in the stenosis ratio. In the FFR measurement, an intravascular pressure measured by a pressure wire is generally used.

For example, as shown in FIG. 4B, the FFR is calculated by indicating a rate of blood flow “Q_{S}” with a stenosis to blood flow “Q_{N}” without a stenosis as a rate of an upstream arterial blood pressure “Pa” of a coronary stenosis Rs to a downstream arterial blood pressure “Pd” of the coronary stenosis Rs, when a myocardial vascular resistance Rm at peak hyperemia is substantially the same. That is, as shown in FIG. 4B, the FFR is calculated as “FFR=Q_{S}/Q_{N}=(Pd/Rm)/(Pa/Rm)=Pd/Pa”. FIGS. 4A and 4B are explanatory diagrams of FFR calculation according to the first embodiment.

Referring back to FIG. 2, for example, it is determined whether the coronary stenosis RS1 and the coronary stenosis RS2 have caused ischemia according to the evaluation method described above, and it is decided to perform the PCI with respect to the stenosis where FFR<0.8. As described above, in the complex usage of indexes, values of various indexes are used. However, these values can be measured in various medical image diagnostic apparatus. Therefore, only by presenting numerals as in the conventional method of utilization, it is troublesome to decide the treatment method and the like by the complex usage of the indexes. Therefore, the medical information processing apparatus 100 according to the first embodiment enables to simplify the complex usage of indexes, thereby supporting the decision of the treatment method by doctors.

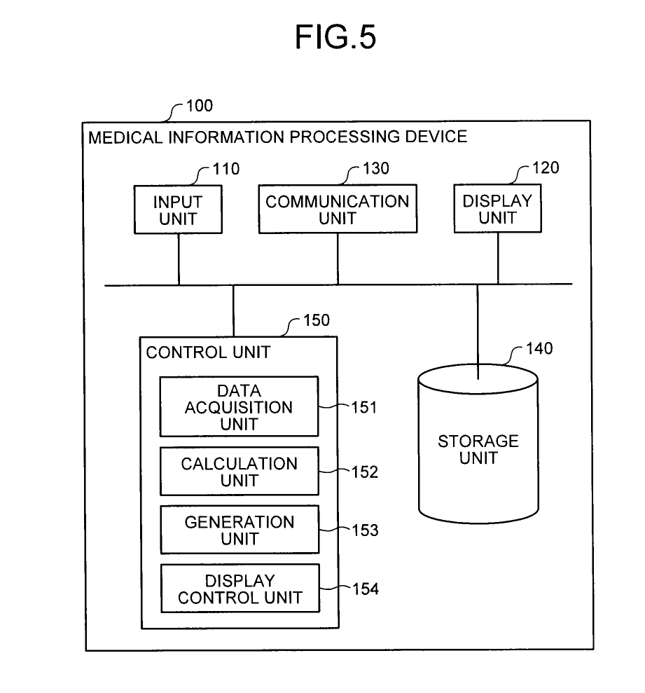

FIG. 5 is an example of a configuration of the medical information processing apparatus 100 according to the first embodiment. As shown in FIG. 5, the medical information processing apparatus 100 includes an input unit 110, a display unit 120, a communication unit 130, a storage unit 140, and a control unit 150. For example, the medical information processing apparatus 100 is a workstation or an arbitrary personal computer, and is connected to the medical image diagnostic apparatus 200 and the image saving apparatus 300 via a network.

The input unit 110 is a mouse, a keyboard, a trackball or the like, and receives an input of various operations with respect to the medical information processing apparatus 100 from an operator (for example, a radiologist). Specifically, the input unit 110 receives an input for acquiring image data and accompanying information associated with a diagnosis of the ischemic cardiac disease, and an input of a specifying operation for specifying an arbitrary region on an image.

The display unit 120 is a liquid crystal panel or the like as a monitor, and displays various types of information. Specifically, the display unit 120 displays a GUI (Graphical User Interface) for receiving various operations from the operator, and display information as a processing result acquired by the control unit 150 (described later). The communication unit 130 is a NIC (Network Interface Card) or the like, and performs communication with other apparatus.

The storage unit 140 is, for example, a semiconductor memory device such as a RAM (Random Access Memory) or a flash memory, or a storage device such as a hard disk or an optical disk, and stores therein image data of medical images acquired by the control unit 150 (described later) and accompanying information. The storage unit 140 also stores therein dominant region information, which is information of a dominant region of the coronary artery. For example, the storage unit 140 stores therein dominant region information, which is information relating to a cardiac muscle region controlled by various blood vessels such as a right coronary artery (RCA), a left anterior descending coronary artery (LAD), and a left circumflex coronary artery (LCX). In other words, the storage unit 140 stores therein information of the feeding vessel for each region of the cardiac muscle.

The control unit 150 is, for example, an electronic circuit such as a CPU (Central Processing Unit) or an MPU (Micro Processing Unit), or an integrated circuit such as an ASIC (Application Specific Integrated Circuit) or an FPGA (Field Programmable Gate Array), and executes overall control of the medical information processing apparatus 100.

As described above, the control unit 150 includes, for example, a data acquisition unit 151, a calculation unit 152, a generation unit 153, and a display control unit 154, and generates and displays display information that enables to simplify complex usage of the plurality of indexes. That is, the control unit 150 generates display information indicating a relative relation between the state of a region including the cardiac muscle (for example, an ischemic state) and the state of a region including a coronal artery (for example, a state of a stenosis), and displays the information.

The data acquisition unit 151 acquires data from the medical image diagnostic apparatus 200 or the image saving apparatus 300 via the communication unit 130. Specifically, the data acquisition unit 151 acquires image data, accompanying information, and index values measured by the medical apparatus from the medical image diagnostic apparatus 200 or the image saving apparatus 300, in response to an instruction received from an operator via the input unit 110, and stores these in the storage unit 140. For example, the data acquisition unit 151 acquires image data of a subject collected by the SPECT scanner for measuring the CFR and image data of the subject collected by the X-ray CT scanner for measuring the FFR. The data acquisition unit 151 also acquires an FFR value of the subject measured by a pressure wire and saved in the image saving apparatus 300.

The calculation unit 152 calculates an index associated with a diagnosis of the ischemic cardiac disease. Specifically, the calculation unit 152 calculates an index in a predetermined region included in the image data acquired by the data acquisition unit 151. The predetermined region included in the image data is specified by various methods. As a first method, a case where all the regions are specified by an operator can be mentioned. That is, the calculation unit 152 calculates an index in a region specified by the operator via the input unit 110, with respect to the image data acquired by the data acquisition unit 151. For example, the calculation unit 152 calculates a CFR value of a region specified in the cardiac muscle included in the image data. When the FFR value has been measured by a pressure wire, the FFR value of the specified region is acquired by the data acquisition unit 151.

Next, as a second method, a case where a region is specified indirectly can be mentioned. For example, the calculation unit 152 respectively calculates an index for a stenosis region present in a coronary artery into which a contrast agent has been injected, and a cardiac muscle region colored by the contrast agent, in an X-ray image taken while injecting the contrast agent into the subject. In this case, for example, by extracting a blood vessel periphery and measuring a diameter of the blood vessel, the calculation unit 152 extracts the stenosis region from the coronary artery into which the contrast agent has been injected. Furthermore, the calculation unit 152 extracts the cardiac muscle region colored by the contrast agent from the image data. The calculation unit 152 calculates the FFR and the CFR for the extracted stenosis region and the cardiac muscle region, respectively. When the FFR value has been measured by a pressure wire, the FFR value of the extracted region is acquired by the data acquisition unit 151. In the example described above, the case of extracting the stenosis region has been explained. However, the operator can specify the region at the time of injecting the contrast agent.

Next, as a third method, a case of using dominant region information stored in the storage unit 140 can be mentioned. In this case, the calculation unit 152 refers to the dominant region information stored in the storage unit 140 to extract the region. For example, when the operator specifies the cardiac muscle region or the stenosis region in a coronary artery, the calculation unit 152 refers to the dominant region information to extract the corresponding coronary artery or cardiac muscle region.

As described above, the calculation unit 152 calculates the respective indexes for the region specified by the various methods. The calculation unit 152 can perform calculation corresponding to respective medical images for the index using the respective medical images. For example, the calculation unit 152 can perform calculation of the CFR using an SPECT image or calculation of the FFR using a CT image. Further, the calculation unit 152 can perform calculation of the CFR by using the SPECT image, the CT image, an MR image, or a PET image. That is, in calculation of the indexes by the calculation unit 152, any method can be applied so long as the respective indexes can be calculated from the image data. In the first embodiment, at the time of calculating the CFR value, the calculation unit 152 calculates a value in each pixel included in the region and designates a mean value of the calculated values as the CFR value of the specified cardiac muscle region.

Referring back to FIG. 5, the generation unit 153 generates display information indicating the state of a first region and the state of a second region according to the CFR of the first region in the subject's cardiac muscle and the FFR of the second region of the feeding vessel in the first region. Specifically, the generation unit 153 generates information indicating the CFR value of the first region and the FFR value of the second region on a graph in which the CFR and the FFR are set respectively on a first axis and a second axis, as the display information.

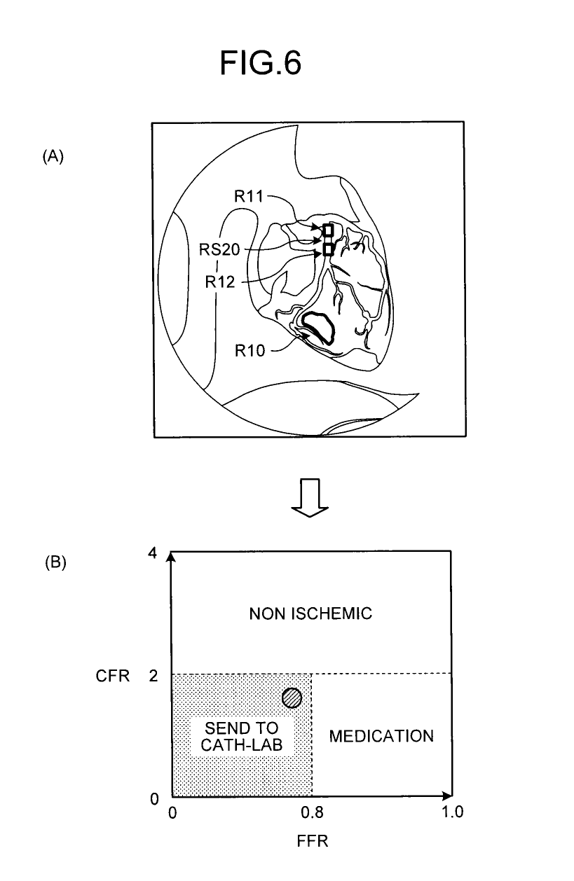

FIG. 6 are examples of generation of display information by the generation unit 153 according to the first embodiment. In FIG. 6, FIG. 6(A) shows a region specified in the image data and FIG. 6(B) shows the display information generated by the generation unit 153. A case where an operator specifies all the regions is explained as an example. For example, on the image shown in FIG. 6(A), when an operator such as a doctor specifies a cardiac muscle region R10, an upstream region R11 of a coronary stenosis RS20 and a downstream region R12 of the coronary stenosis RS20, the data acquisition unit 151 acquires image data for calculating the index in each region.

The calculation unit 152 extracts the specified region in the acquired image data to calculate the index in each extracted region. For example, the calculation unit 152 calculates the CFR of a region corresponding to the region R10 in the SPECT image. The calculation unit 152 also calculates the FFR in the coronary stenosis RS20 from regions corresponding to the regions R11 and R12 in the CT image. Extraction of the region corresponding to the specified region in the respective pieces of image data can be performed by using existing techniques such as a method of using atlas data or the like. When the FFR in the coronary stenosis RS20 has been measured by a pressure wire, the data acquisition unit 151 acquires the measurement value and notifies the calculation unit 152 of the measurement value.

When the CFR and the FFR are calculated by the calculation unit 152, the generation unit 153 generates a graph in which the FFR is set on a horizontal lower axis, as shown in FIG. 6(B), and the CFR is set on a longitudinal left axis, and generates display information in which a point is arranged at a position calculated by the calculation unit 152.

The generation unit 153 divides the graph to regions for each treatment content decided based on a threshold set to the index respectively set on each axis. That is, as shown in FIG. 6(B), the generation unit 153 divides the graph by a threshold “2” of the CFR and a threshold “0.8” of the FFR, and allocates a treatment content to each region. For example, as shown in FIG. 6(B), “send to cath-Lab” that means sending a treatment content to a catheter operation room (implementation of PCI) is allocated to a region of “CFR<2, FFR<0.8”, “Medication” that means implementation of medication is allocated to a region of “CFR<2, FFR>0.8”, and “Non ischemic” that means no ischemia is allocated to a region of “CFR>2”.

In the above examples, a case where the graph is generated after the CFR and the FFR are calculated by the calculation unit 152 has been explained. However, the embodiment is not limited thereto, and such a case can be considered that a graph is generated beforehand and stored in the storage unit 140, and when the CFR and the FFR are calculated by the calculation unit 152, the generation unit 153 reads the graph and generates the display information in which the calculation result is plotted on the read graph.

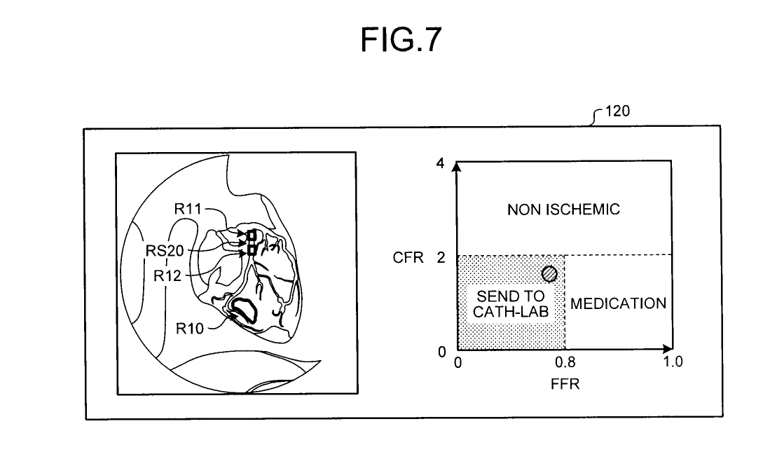

Referring back to FIG. 5, the display control unit 154 executes control such that the display information generated by the generation unit 153 is shown by the display unit 120. FIG. 7 is an example of information displayed under control of the display control unit 154 according to the first embodiment. For example, the display control unit 154 causes the display unit 120 to display an image of the heart, whose region is specified, and a display image generated by the generation unit 153 parallel to each other. Accordingly, an operator can judge at one view that the PCI is implemented with respect to the coronary stenosis RS20 as an effective treatment method for ischemia in the specified region R10.

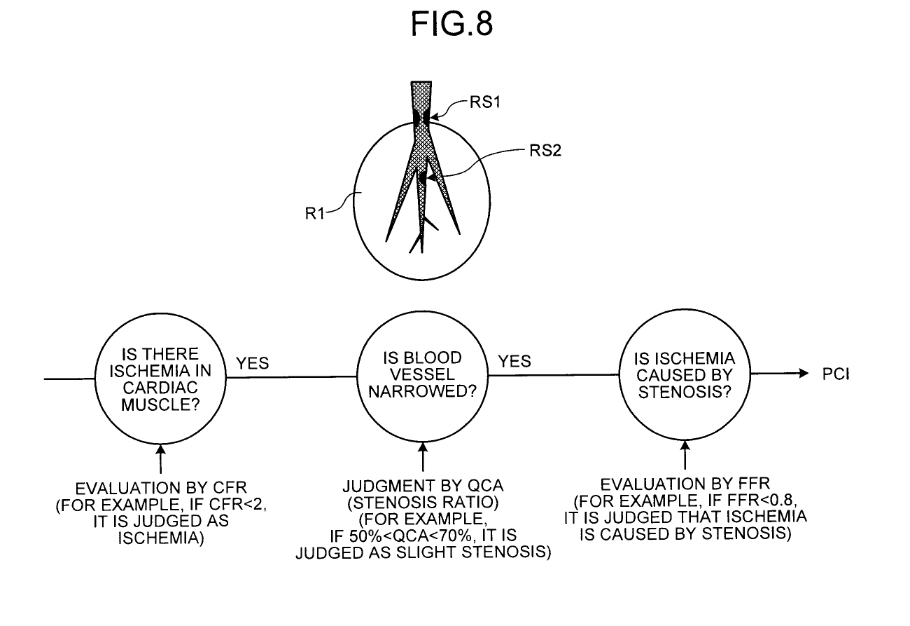

In the above examples, a case of using the CFR and the FFR as the index has been explained. However, in the medical information processing apparatus 100 according to the first embodiment, the number of indexes can be further increased. For example, at the time of determining whether to perform the PCI, the stenosis ratio of the coronary stenosis may be used. Therefore, a case where the stenosis ratio is further added as complex usage of the plurality of indexes is explained below. FIG. 8 is an explanatory diagram of a second example of complex usage of a plurality of indexes according to the first embodiment.

In FIG. 8, a determination of the stenosis ratio is added to the example shown in FIG. 2. That is, as shown in FIG. 8, a decision of a treatment method of performing PCI with respect to a stenosis site can be mentioned, when there is ischemia in the cardiac muscle and also a stenosis has occurred, and the ischemia is caused by the stenosis. As an example, as shown in FIG. 8, it is determined whether the blood vessel is narrowed when it is determined that there is the ischemia by the evaluation by the CFR.

It is evaluated whether the blood vessel is narrowed by a QCA (Quantitative Coronary Analysis). As the evaluation, for example, if “50%<QCA<70%”, it is determined that a slight stenosis has occurred. The QCA is an index indicating what percent of stenosis has occurred (the stenosis ratio) quantitatively, and is calculated by using an X-ray image to measure the diameter of the blood vessel. When the stenosis ratio is very high (in a case of a severe stenosis), the PCI is implemented, and when the stenosis ratio is very low (in a case of a mild stenosis), the PCI does not need to be implemented. Therefore, when a slight stenosis has occurred as described above, it is determined whether to perform the PCI.

For example, when the stenosis ratio of the coronary stenosis RS1 or the coronary stenosis RS2 shown in FIG. 8 is “50% to 70%”, an FFR determination is then performed whether the coronary stenosis RS1 or the coronary stenosis RS2 results in the ischemia. In this manner, the medical information processing apparatus 100 according to the first embodiment enables to simplify the complex usage of three or more indexes. An example of complex usage of three indexes is explained below.

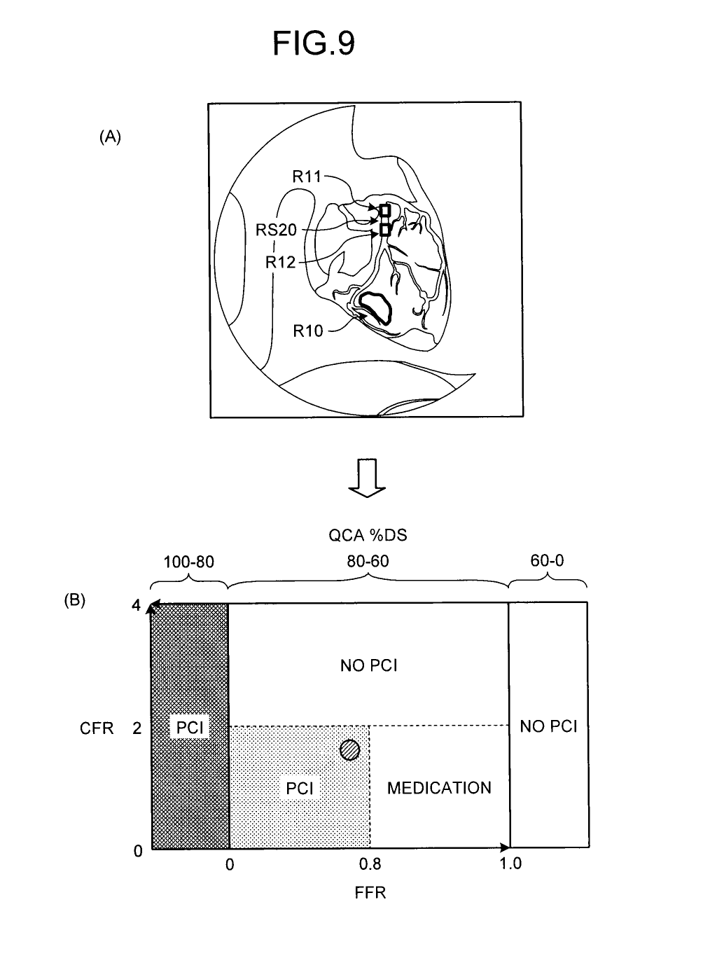

FIG. 9 are examples of generation of display information by the generation unit according to the first embodiment. In FIG. 9, FIG. 9(A) shows a region specified on image data and FIG. 9(B) shows display information generated by the generation unit 153. A case where an operator specifies all the regions is explained as an example. For example, on the image shown in FIG. 9(A), when an operator such as a doctor specifies the cardiac muscle region R10, the upstream region R11 of the coronary stenosis RS20 and the downstream region R12 of the coronary stenosis RS20, the data acquisition unit 151 acquires image data for calculating the index in each region.

The calculation unit 152 calculates the stenosis ratio of the coronary stenosis RS20 in addition to the calculation of the CFR and the FFR. The generation unit 153 generates a graph in which the QCA (% DS) is set on a horizontal upper axis, as shown in FIG. 9(B), in addition to the setting of the FFR and the CFR on the horizontal lower axis and the longitudinal left axis, and arranges a point at a position calculated by the calculation unit 152 in the generated graph.

The generation unit 153 sets the axis so that the range in which the QCA (% DS) becomes “80-60” corresponds to the region where the determination of the CFR and the FFR is performed. As shown in FIG. 9(B), the generation unit 153 allocates “PCI” that means implementation of the PCI to a region in which the QCA (% DS) becomes “100-80”, and “no PCI” that means the PCI is not implemented to a region in which the QCA (% DS) becomes “60-0”.

As shown in FIG. 9(B), the generation unit 153 generates display information in which “PCI” that means implementation of the PCI is allocated to a region of “CFR<2, FFR<0.8”, “Medication” that means implementation of medication is allocated to a region of “CFR<2, FFR>0.8”, and “no PCI” that means the PCI is not implemented is allocated to a region of “CFR>2”, in the range in which the QCA (% DS) becomes “80-60”. As described above, the medical information processing apparatus 100 according to the first embodiment enables to simplify the complex usage of three or more indexes. That is, by displaying the display information as shown in FIG. 9(B) by the display unit 120, an operator such as a doctor can judge at one view an effective treatment method for the ischemia using three of more indexes.

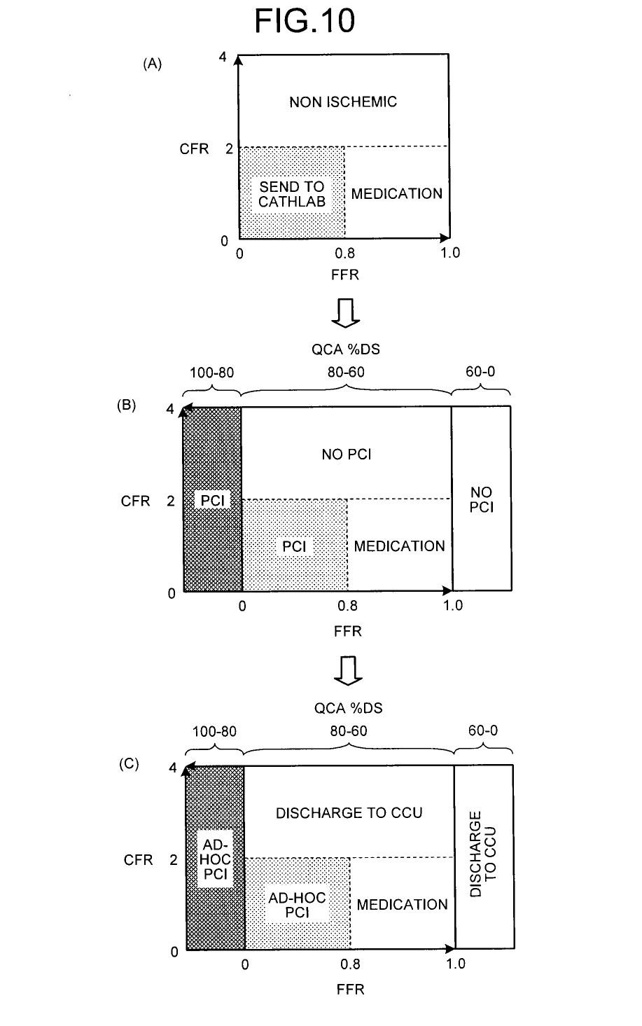

In the graphs as shown in FIG. 6 or 9, the treatment contents to be allocated, the indexes to be used, and the threshold of the index can be arbitrarily set by an operator. For example, the treatment contents and the index can be changed depending on the process of the diagnostic treatment. FIG. 10 are examples of a graph to be generated by the generation unit 153 according to the first embodiment. In FIG. 10, a graph used at a stage of a diagnosis and planning of treatment is shown in FIG. 10(A), a graph used at a stage before implementation of the PCI is shown in FIG. 10(B), and a graph used at a stage after implementation of the PCI is shown in FIG. 10(C).

For example, at the stage of a diagnosis and planning of treatment, the generation unit 153 generates a graph in which the CFR and the FFR are plotted on the axes, as shown in FIG. 10(A). At the stage before implementation of the PCI, as shown in FIG. 10(B), the generation unit 153 generates a graph in which the CFR, the FFR, and the QCA are plotted on the axes, to allocate whether to perform the PCI or to perform medication. At the stage after implementation of the PCI, as shown in FIG. 10(C), the generation unit 153 generates a graph in which the CFR, the FFR, and the QCA are plotted on the axes, to allocate implementation of Ad-hoc PCI, implementation of medication, and discharge to a coronary care unit.

As described above, in the graph generated by the generation unit 153, the treatment contents and the index can be set arbitrarily. However, for example, the graph can be switched depending on not only the process of diagnostic treatment described above, but also the index that can be calculated (acquired).

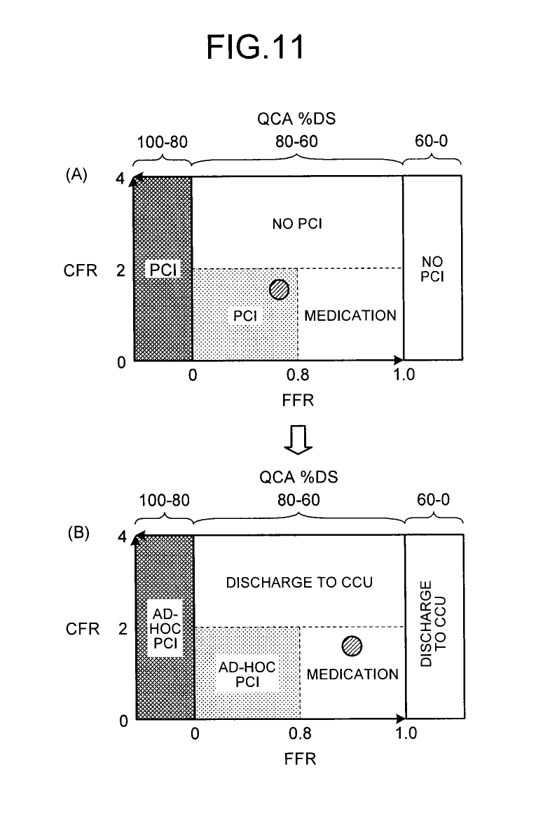

Furthermore, by switching the graph for each process of diagnostic treatment, a detailed determination based on various statuses can be performed and how the subject changes (is improved) before and after the treatment can be confirmed at one view. FIG. 11 are examples of display information to be display-controlled by the display control unit 154 according to the first embodiment. In FIG. 11, a graph at a stage before implementation of the PCI is shown in FIG. 11(A), and a graph at a stage after implementation of the PCI is shown in FIG. 11(B).

For example, by displaying graphs generated at respective stages by the generation unit 153 in order of FIG. 11(A) and FIG. 11(B) (in chronological order), the display control unit 154 enables an operator to ascertain at one view that the degree of the stenosis has been alleviated (the FFR value has increased) by implementation of the PCI, and the subject only needs the medication. The graphs in FIG. 11(A) and FIG. 11(B) can be displayed in parallel.

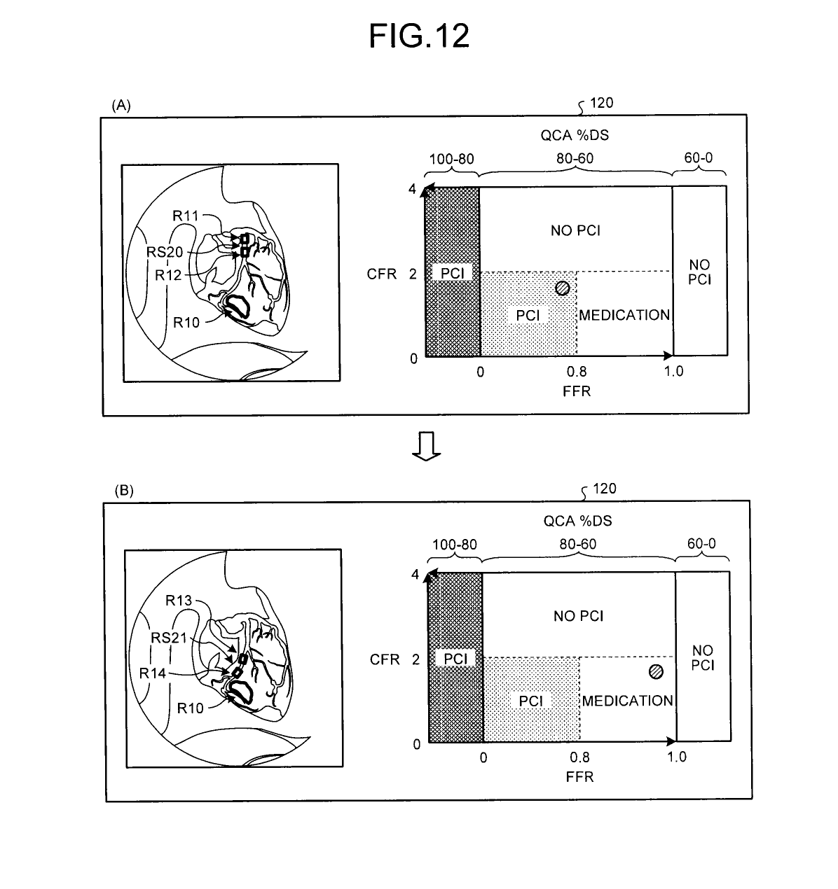

The medical information processing apparatus 100 according to the first embodiment can arbitrarily change the region specified in the image data to generate display information following the change, and display the generated display information. That is, the input unit 110 receives an instruction to change the value for at least one of the CFR value, the FFR value, and the QCA value. The generation unit 153 regenerates the display information in which the value in response to the change instruction received by the input unit 110 is shown. The display control unit 154 executes control such that the display information regenerated by the generation unit 153 is shown by the display unit 120. FIG. 12 are examples of a change of display information, which follows a region change by the medical information processing apparatus 100 according to the first embodiment. In FIG. 12, display contents before the region change are shown in FIG. 12(A), and display contents after the region change are shown in FIG. 12(B).

For example, as shown in FIG. 12(A), it is assumed that the region R10, and the upstream region R11 and the downstream region R12 of the coronary stenosis RS20 are specified before the region change, and a graph of the CFR, the FFR, and the QCA corresponding to these regions is displayed. The input unit 110 can receive an instruction to change each region on the image. For example, as shown in FIG. 12(B), when the coronary stenosis RS20, the upstream region R11 thereof, and the downstream region R12 thereof are changed to a coronary stenosis RS21, an upstream region R13 thereof, and a downstream region R14 thereof, the calculation unit 152 calculates or acquires the respective indexes (FFR and QCA) of the changed region.

The generation unit 153 regenerates display information by using the index value calculated or acquired by the calculation unit 152. For example, the generation unit 153 generates a graph in which the position of a plot has been changed, as shown in the right graph in FIG. 12(B). The display control unit 154 causes the display unit 120 to display the display information generated by the generation unit 153 and the image. The medical information processing apparatus 100 according to the first embodiment performs the process described above as a background process, and when the region is changed on the image, generates a graph corresponding to the change, and displays the graph. Accordingly, the display information in which the plot on the graph is changed following the region change can be provided to an operator.

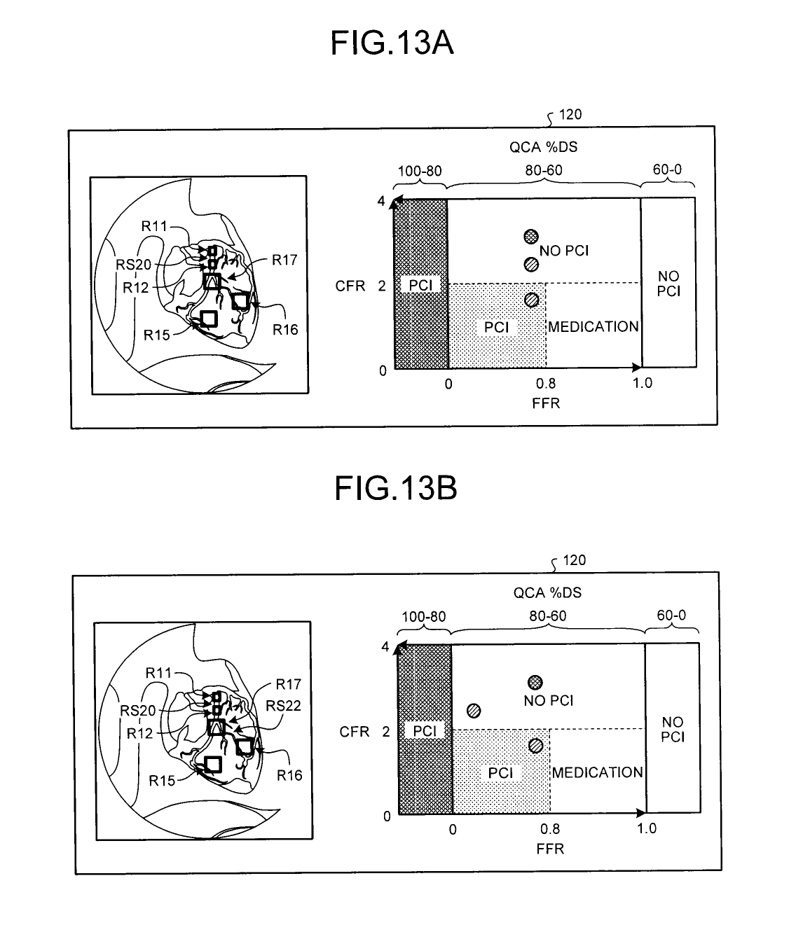

A case where a result of a determination of the relative relation between single regions is plotted on a graph has been explained above. However, the medical information processing apparatus 100 according to the first embodiment can plot a result relating to a plurality of regions on a graph simultaneously. The case of using a plurality of regions is explained below with reference to FIG. 13A and FIG. 13B. FIG. 13A and FIG. 13B are display examples of display information according to the first embodiment.

For example, the medical information processing apparatus 100 generates display information in which results of determinations of relative relations between a plurality of regions in the cardiac muscle with respect to a single stenosis site are plotted on a graph, and displays the generated display information. As an example, in the medical information processing apparatus 100, as shown in FIG. 13A, the calculation unit 152 calculates the respective CFR values of cardiac muscle regions R15, R16, and R17, the QCA (the stenosis ratio) in the coronary stenosis RS20, and the FFR using the upstream region R11 and the downstream region R12 thereof. The generation unit 153 associates the calculation results of the QCA and the FFR with the respective CFR values of the region R15, the region R16, and the region R17 calculated by the calculation unit 152, to generate display information in which the association result is plotted on a graph. That is, the generation unit 153 generates a graph in which three points having different CFR values are plotted as shown on the right graph in FIG. 13A.

Furthermore, for example, the medical information processing apparatus 100 generates display information in which results of determinations of relative relations between the regions in the cardiac muscle and a plurality of stenosis sites are plotted on a graph and displays the generated display information. As an example, in the medical information processing apparatus 100, as shown in FIG. 13B, the calculation unit 152 calculates the respective CFR values of the cardiac muscle regions R15, R16, and R17, the QCA (the stenosis ratio) in the coronary stenosis RS20, and the FFR using the upstream region R11 and the downstream region R12 thereof. The calculation unit 152 also calculates the QCA in a coronary stenosis RS22, and the FFR using the upstream region and the downstream region thereof.

The generation unit 153 associates the calculation results of the QCA in the coronary stenosis RS20, and the FFR using the upstream region R11 and the downstream region R12 thereof with the respective CFR values of the region R15 and the region R17 calculated by the calculation unit 152, to plot these on a graph. Further, the generation unit 153 also associates the calculation results of the QCA in the coronary stenosis RS22 and the FFR using the upstream and downstream regions thereof with the CFR of the region R16 and plots the association result on a graph. That is, as shown in the right graph of FIG. 13B, the generation unit 153 generates a graph in which three spots having different values of the CFR and the FFR are plotted.

As described above, the medical information processing apparatus 100 according to the first embodiment can generate the display information in which a plurality of points are plotted on a graph. The index relating to a diagnosis of the ischemic cardiac disease can be acquired by a plurality of apparatus, as described above. For example, the CFR can be acquired from the SPECT image, the CT image, the MR image, and the PET image. The CFR value acquired from these images may be different respectively. Therefore, the medical information processing apparatus 100 according to the first embodiment generates and displays display information that indicates by which apparatus respective index values are acquired.

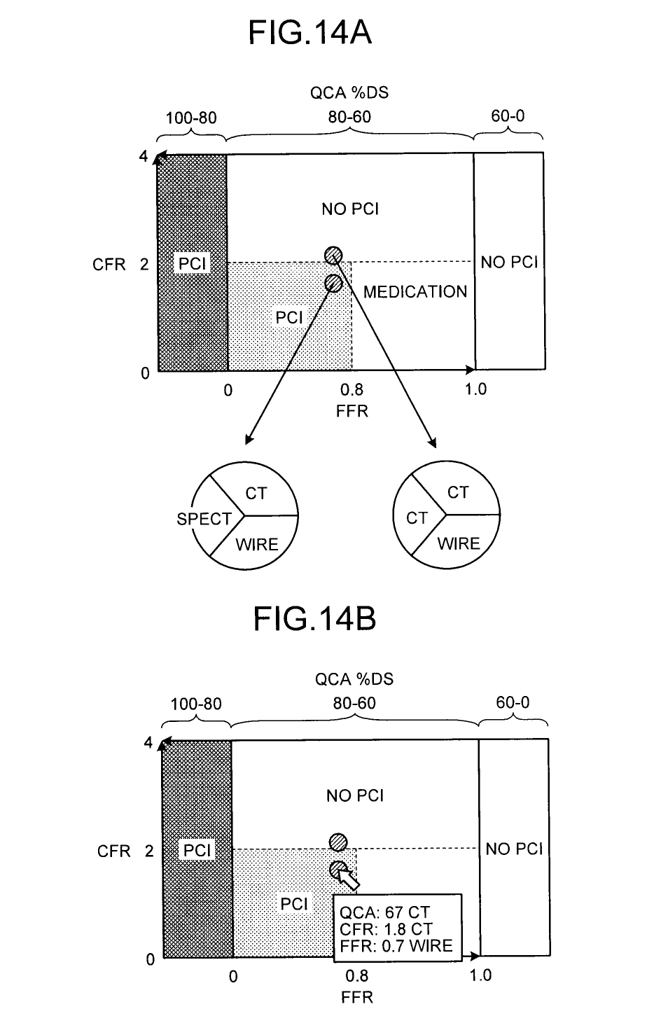

FIG. 14A is an example of display information generated by the medical information processing apparatus 100 according to the first embodiment. In FIG. 14A, a diagram in which the plot on the graph is shown in an enlarged scale. For example, as shown in FIG. 14A, the medical information processing apparatus 100 generates and displays display information indicating the apparatus that has calculated (acquired) the respective index values in the plot. In this case, the generation unit 153 receives information of the modality or medical apparatus that has collected the image data used for calculation of the index by the calculation unit 152, and generates a plot reflecting the received information.

For example, as shown in FIG. 14A, the generation unit 153 generate a plot indicating that the CFR is calculated from the SPECT image, the QCA is calculated from the CT image, and the FFR is acquired from the value of a pressure wire. Furthermore, the generation unit 153 generates a plot indicating that the CFR in the same region is calculated from the CT image. The index and the position in the plot are associated with each other beforehand, and can be set arbitrarily by an operator. The generation unit 153 then generates display information in which the generated plot is arranged on a graph, and the display control unit 154 causes the display unit 120 to display the information. Accordingly, the medical information processing apparatus 100 according to the first embodiment can provide the display information that enables the operator to consider a calculation or acquisition method of the respective indexes.

Further, the medical information processing apparatus 100 according to the first embodiment can display an apparatus that has performed a measurement for each index and a measurement value thereof, for a plot specified by the operator. FIG. 14B is another example of display information generated by the medical information processing apparatus 100 according to the first embodiment. For example, as shown in FIG. 14B, the medical information processing apparatus 100 displays the information of the apparatus that has performed a measurement for each index and the measurement value thereof additionally on a graph. As an example, when the operator operates a mouse to place a pointer on a plot, information as shown in FIG. 14B is displayed.

In this case, the generation unit 153 receives the information of the modality or medical apparatus that has collected the image data used for calculation of the index by the calculation unit 152 for each plot, and generates information reflecting the received information. For example, as shown in FIG. 14B, the generation unit 153 generates information indicating that a lower plot on the graph is “QCA, value: 67, apparatus: CT”, “CFR, value: 1.8, apparatus: CT”, and “FFR, value: 0.7, apparatus: wire”. The display control unit 154 then displays the generated information of the plot instructed by the pointer of the mouse on the display unit 120. The information shown in FIG. 14B can be generated beforehand, or can be generated in real time by the generation unit 153 when the plot is indicated by the pointer.

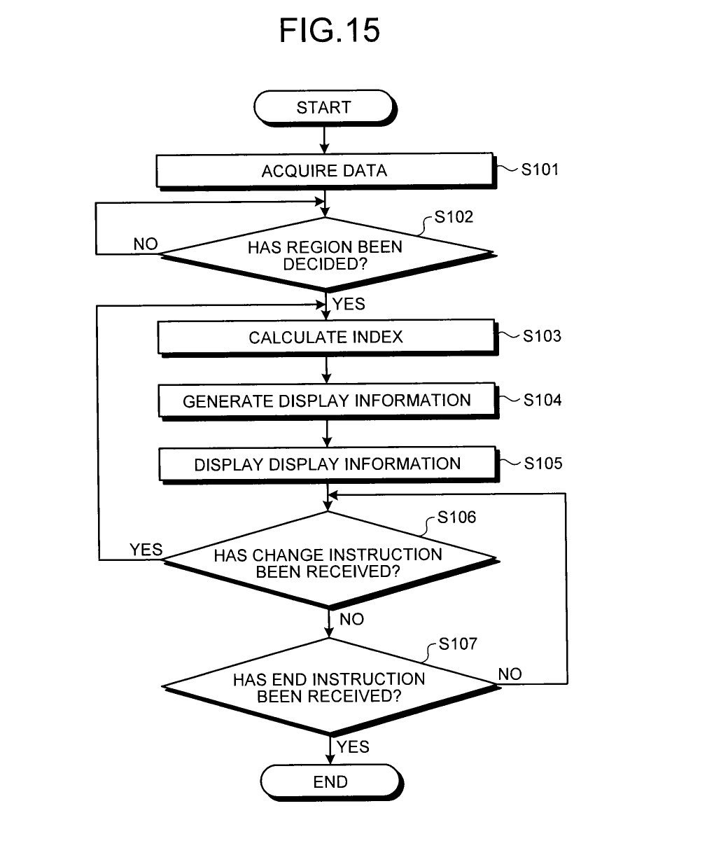

A process procedure of the medical information processing apparatus 100 according to the first embodiment is explained next with reference to FIG. 15. FIG. 15 is a flowchart of a process procedure performed by the medical information processing apparatus 100 according to the first embodiment. In FIG. 15, a process after image data is collected in the medical image diagnostic apparatus 200 is shown.

As shown in FIG. 15, in the medical information processing apparatus 100 according to the first embodiment, the data acquisition unit 151 acquires data such as image data, accompanying information, and measurement results measured by the medical apparatus (Step S101), and determines whether a region on the display image has been decided (Step S102). When a region is decided (YES at Step S102), the calculation unit 152 calculates the index in the decided region (Step S103). The medical information processing apparatus 100 is in a standby state until the region is decided (NO at Step S102).

When the index is calculated, the generation unit 153 generates display information (Step S104), and the display control unit 154 caused the display unit 120 to display the generated display information (Step S105). The calculation unit 152 determines whether a region change instruction has been received (Step S106). When the region change instruction has been received (YES at Step S106), the calculation unit 152 returns to Step S103 to calculate the index after the region is changed.

On the other hand, when the region change instruction has not been received (NO at Step S106), the medical information processing apparatus 100 determines whether an end instruction has been received (Step S107). When it is determined that the end instruction has not been received (NO at Step S107), control return to Step S106, and the calculation unit 152 performs the determination process. On the other hand, when it is determined that the end instruction has been received (YES at Step S107), the medical information processing apparatus 100 finishes the process.

As described above, according to the first embodiment, the generation unit 153 generates display information indicating the state of the first region and the state of the second region depending on the CFR of the first region in the subject's cardiac muscle and the FFR of the second region of the feeding vessel in the first region. The display control unit 154 executes control such that the display information generated by the generation unit 153 is shown by the display unit 120. Consequently, the medical information processing apparatus 100 according to the first embodiment can visually display the relative relation of the states of each of regions indicated respectively by the CFR and the FFR, and enables to simplify complex usage of the plurality of indexes.

According to the first embodiment, the generation unit 153 generates information in which the CFR value of the first region and the FFR value of the second region are plotted on a graph in which the CFR and the FFR are respectively set on the first axis and the second axis, as display information. Consequently, the medical information processing apparatus 100 according to the first embodiment can display the relative relation of the states of the regions respectively indicated by the CFR and the FFR in a format easily understandable by an operator.

According to the first embodiment, the generation unit 153 generates information indicating the CFR value of the first region, the FFR value of the second region, and the stenosis ratio (QCA) of a stenosis included in the second region on a graph in which the stenosis ratio of the stenosis included in the second region is set on a third axis in addition to the CFR and the FFR, as display information. Consequently, the medical information processing apparatus 100 according to the first embodiment enables to simplify the complex usage of the plurality of indexes even when the judgment standard is set more finely by using three or more indexes.

According to the first embodiment, the input unit 110 receives a value change instruction for at least one of the CFR value, the FFR value, and the QCA value. The generation unit 153 regenerates display information in which a value corresponding to the change instruction received by the input unit 110 is shown. The display control unit 154 executes control such that the display information regenerated by the generation unit 153 is shown by the display unit 120. Consequently, the medical information processing apparatus 100 according to the first embodiment can immediately show the display information reflecting the index state desired by an operator, and can improve the test accuracy.

According to the first embodiment, the generation unit 153 generates information in which values acquired by a plurality of different apparatus are shown on a graph respectively for at least one of the CFR value, the FFR value, and the value of the stenosis ratio (QCA) included in the second region, as display information. Consequently, the medical information processing apparatus 100 according to the first embodiment can present respective results for the index whose value changes because of using different acquisition apparatus, and enables for an operator to adapt to circumstances.

According to the first embodiment, the generation unit 153 generates display information in which the graph is divided to regions for each treatment content decided based on the threshold set to each index respectively set on each axis. Consequently, the medical information processing apparatus 100 according to the first embodiment enables an operator to ascertain the treatment content at one view.

According to the first embodiment, the input unit 110 receives a change instruction of the threshold set to each index respectively set on each axis. The generation unit 153 sets a threshold in response to the change instruction received by the input unit 110 to each axis and generates display information in which a graph is divided to regions for each treatment content based on the set threshold. Consequently, the medical information processing apparatus 100 according to the first embodiment enables to respond to fine requests of an operator in real time.

### Second Embodiment

In the first embodiment described above, a case where a graph is generated and displayed as display information has been explained. In a second embodiment, a case of generating and displaying an image in which a cardiac muscle image and a coronary artery image are color mapped as display information is explained. In the medical information processing apparatus 100 according to the second embodiment, the processing contents of the generation unit 153 and the display control unit 154 are different from those of the medical information processing apparatus 100 according to the first embodiment. This point is mainly explained below.

The generation unit 153 according to the second embodiment generates, as display information, a composite image in which a first image obtained by color mapping the cardiac muscle image of the subject in a color corresponding to the CFR value of the first region, and a second image obtained by color mapping an image of a feeding vessel in a color corresponding to the FFR value of the second region are shown on the same image. Specifically, the generation unit 153 generates the first image by color mapping respective pixels in the cardiac muscle image used for calculation of the CFR by the calculation unit 152 in a color corresponding to the CFR value.

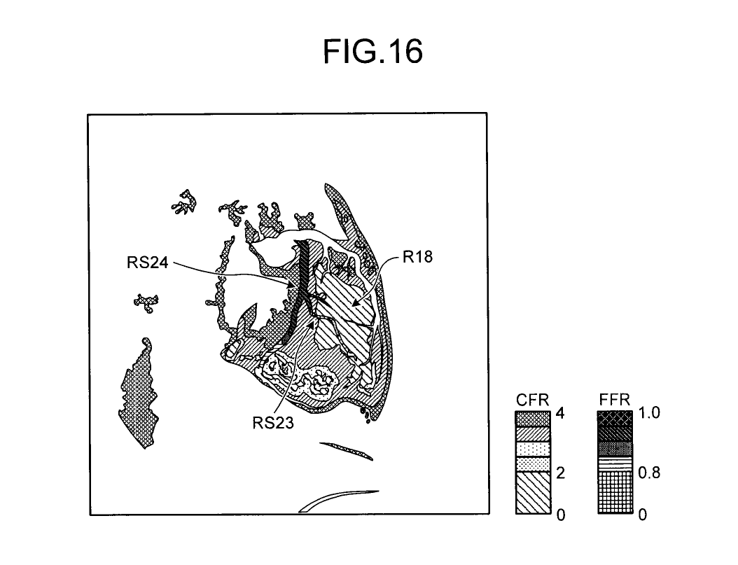

Similarly, the generation unit 153 generates the second image by color mapping the coronary artery image used for calculation of the FFR by the calculation unit 152 in a color corresponding to the FFR value. The generation unit 153 divides the coronary artery by the stenosis region, and colors the divided regions by a color corresponding to the FFR value of the stenosis in the region. FIG. 16 is an example of display information generated by the generation unit 153 according to the second embodiment.

For example, as shown in FIG. 16, the generation unit 153 generates display information in which the cardiac muscle image including a region R18 and the coronary artery image including a coronary stenosis RS2 and a coronary stenosis RS24 are color-mapped based on the CFR value and the FFR value. Therefore, by displaying the image as shown in FIG. 16 on the display unit 120 by the display control unit 154, an operator (an observer) can understand immediately that for example, ischemia has occurred in the region R18 and the stenosis that results in the ischemia is the coronary stenosis RS23.

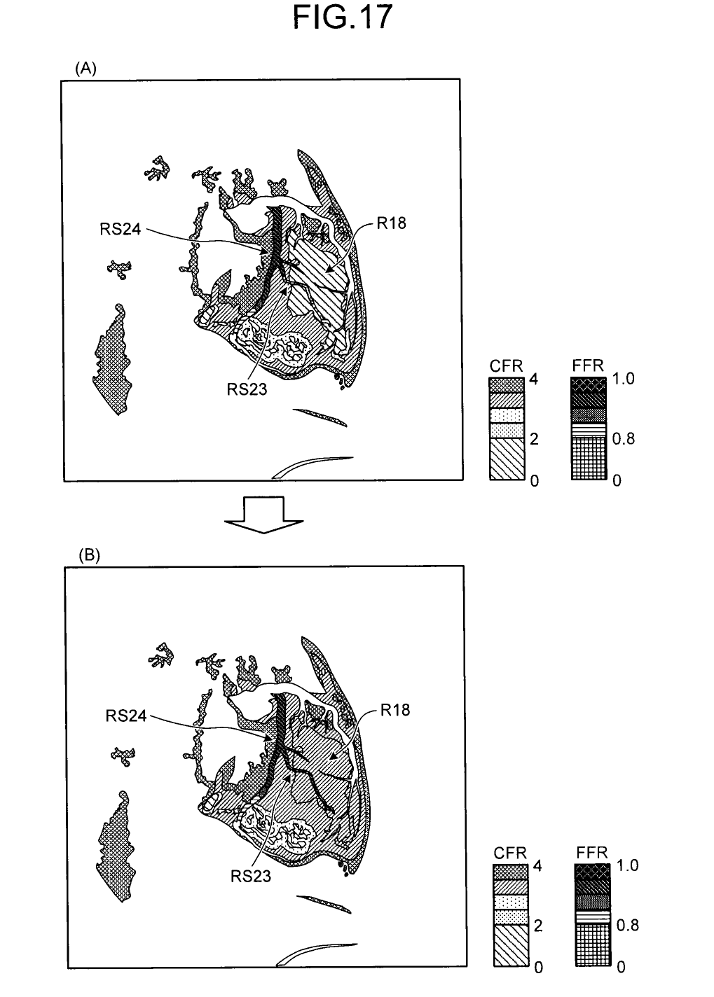

FIG. 17 are examples of information displayed under control of the display control unit according to the second embodiment. In FIG. 17, an image before implementation of the PCI is shown in FIG. 17(A) and an image after implementation of the PCI is shown in FIG. 17(B). For example, a doctor judges that ischemia has occurred in the region R18 and the coronary stenosis RS23 is the cause thereof, by referring to the image shown in FIG. 17(A). As a result, the doctor implements the PCI with respect to the coronary stenosis RS23.

Thereafter, the medical information processing apparatus 100 regenerates and displays an image of the same patient as shown in FIG. 17(B). A doctor can immediately confirm that the ischemia in the region R18 has been improved by referring to the image shown in FIG. 17(B). In the above examples, a case of performing color mapping corresponding to the CFR value and the FFR value has been explained. However, the embodiment is not limited thereto, and for example, only a region of CFR<2 and a region of FFR<0.8 can be colored. Further, the color for coloring each region can be arbitrarily set, and for example, the CFR and FFR can be respectively expressed with a contrasting density of the same type of color. Further, a color can be allocated to each of treatment contents and coloring corresponding to the treatment contents can be performed with respect to the cardiac muscle region and the stenosis region. For example, the CFR value and the FFR value of the region can be displayed by applying a pointer to the coronary stenosis region and the ischemia region.

As described above, according to the second embodiment, a composite image in which the first image obtained by color mapping the cardiac muscle image of the subject in a color corresponding to the CFR value of the first region, and the second image obtained by color mapping a feeding vessel image in a color corresponding to the FFR value of the second region are shown on the same image is generated as the display information. Accordingly, the medical information processing apparatus 100 according to the second embodiment enables an operator (an observer) to ascertain the state of ischemia in the cardiac muscle and the position of the stenosis that has caused the ischemia immediately.

### Third Embodiment

While the first and second embodiments have been explained above, the present application can be carried out by various different modes other than the first and second embodiments.

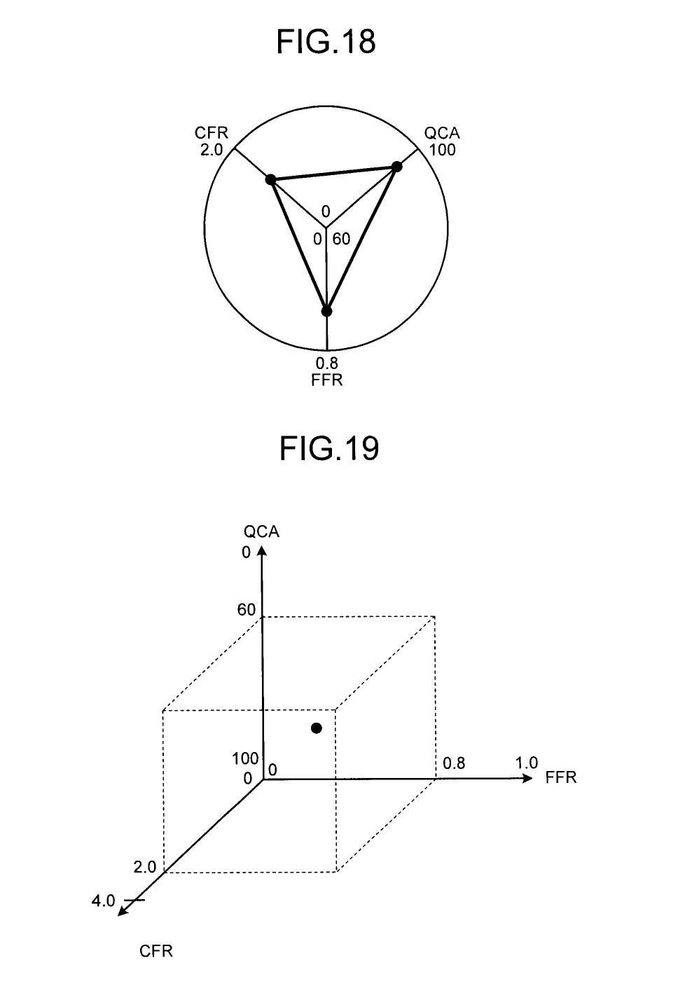

In the first embodiment described above, the case of generating the graph in which the FFR is set on the horizontal lower axis, the QCA is set on the horizontal upper axis, and the CFR is set on the longitudinal left axis has been explained. However, the embodiments are not limited thereto, and an arbitrary graph can be generated. FIGS. 18 and 19 are examples of display information to be displayed by the medical information processing apparatus 100 according to a third embodiment.

The medical information processing apparatus 100 according to the third embodiment can generate and display a radar chart, as shown in FIG. 18. In this case, for example, as shown in FIG. 18, the generation unit 153 generates a radar chart in which values of the CFR, FFR, and QCA calculated by the calculation unit 152 are plotted on respective axes, as display information. The values of the respective axes can be set arbitrarily.

Furthermore, the medical information processing apparatus 100 according to the third embodiment can generate and display a graph of XYZ coordinates, as shown in FIG. 19. In this case, for example, as shown in FIG. 19, the generation unit 153 generates a graph in which the FFR, CFR, and QCA are respectively set on XYZ axes, generates a region bounded by thresholds on respective axes, and shows the region in the graph. The generation unit 153 generates a graph plotted at positions corresponding to the CFR value, the FFR value, and the QCA value calculated by the calculation unit 152 as display information.

The thresholds set on respective axes can be arbitrarily set. The region bounded by thresholds on the respective axes shown in FIG. 19 is shown, for example, in a translucent state by increasing a permeation rate. Further, the region bounded by thresholds on the respective axes shown in FIG. 19 can be colored.

In the first and second embodiments described above, a case where the calculation unit 152 of the medical information processing apparatus 100 calculates the respective index values by using image data has been explained. However, the embodiments are not limited thereto, and for example, index values calculated by respective modalities can be used.

In this case, the data acquisition unit 151 acquires the image data and accompanying information thereof, to acquire image data in which the index values have been calculated, and the calculated index values. The generation unit 153 uses the respective index values acquired by the data acquisition unit 151 to generate display information such as a graph or an image. The display control unit 154 causes the display unit 120 to display the generated display information.

As described above, the medical information processing apparatus 100 according to the present application generates display information by using index values calculated from the image data by the calculation unit 152 or respective modalities and index values measured by the medical apparatus, and causes the display unit 120 to display the generated display information. As the display information displayed by the display unit 120, information in which a point is arranged at a position corresponding to the index value selected at the present moment is generated. For example, as explained with reference to FIG. 12, when the input unit 110 receives a change instruction of a region on an image, the medical information processing apparatus 100 calculates an index value following the change to generate display information, and displays the generated display information by the display unit 120.

The medical information processing apparatus 100 according to the present application can receive various change instructions, other than the example of the region change on the image described above, and display the display information in response to the change instruction. For example, the medical information processing apparatus 100 receives an instruction to change to another medical image collected from the same patient and select a region on the image, calculates or acquires index values corresponding to the received instruction to generate display information, and displays the generated display information. Furthermore, the input unit 110 of the medical information processing apparatus 100 can receive a direct input operation of an index value. As an example, the display unit 120 displays a GUI for inputting figures of the CFR and FFR, and the input unit 110 receives the input of figures. The generation unit 153 generates display information corresponding to the figures received by the input unit 110, and the display control unit 154 shows the generated display information on the display unit 120.

In this manner, the medical information processing apparatus 100 generates display information in which a point is arranged at a position corresponding to the index value selected at the present moment and shows the display information by the display unit 120. At this time, for example, as explained with reference to FIG. 14A or 14B, the medical information processing apparatus 100 generates and displays display information indicating an apparatus that has calculated (acquired) the index value used for the display information at the present moment. That is, the medical information processing apparatus 100 generates and displays display information indicating an apparatus that has calculated (acquired) the index value inside the plot on the graph, or additionally displays display information indicating the apparatus that has calculated (acquired) the index value used for the display information, for the plot indicated by a mouse pointer.

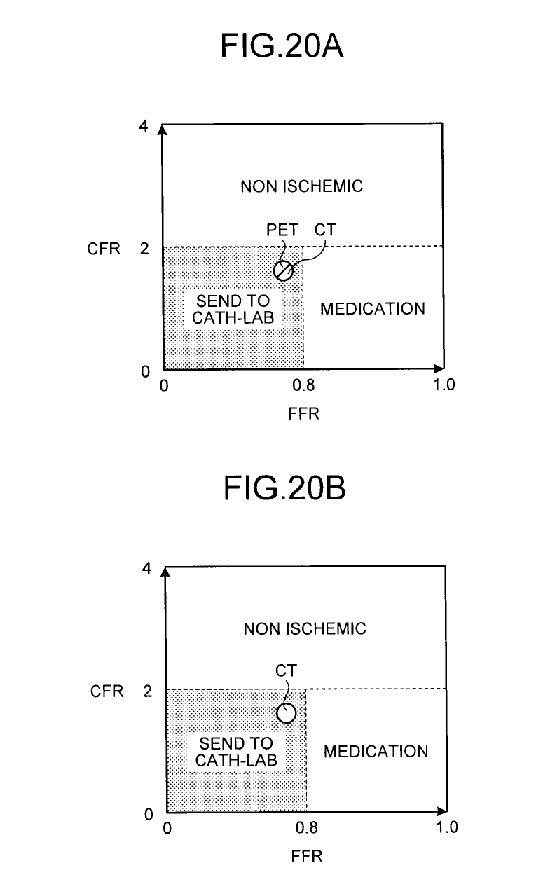

In FIGS. 14A and 14B, an example in which three values of the FFR, CFR, and QCA are calculated (acquired) is shown. However, the embodiments are not limited thereto, and display information can be generated and displayed in a similar manner in a case where the index values are other than those. FIGS. 20A and 20B are examples of display information to be displayed by the medical information processing apparatus 100 according to the third embodiment.

For example, as shown in FIG. 20A, when display information indicating the states of the CFR and FFR are shown, the medical information processing apparatus 100 shows display information indicating a calculation (acquisition) apparatus inside the plot. In FIG. 20A, two drawing lines are drawn from inside the plot, and “PET” and “CT” are respectively shown. However, in practice, “PET” and “CT” are shown inside the plot. That is, in the plot shown in FIG. 20A, “PET” is shown on the left of a diagonal line, and “CT” is shown on the right thereof. This means that the CFR is calculated (acquired) from “PET image”, and the FFR is calculated (acquired) from “CT image”.

Furthermore, when two indexes are calculated (acquired) by the same apparatus, as shown in FIG. 20B, the medical information processing apparatus 100 shows display information in which a single apparatus is shown inside the plot. For example, as shown in FIG. 20B, the medical information processing apparatus 100 shows display information in which “CT” is shown inside the plot on a graph of the CFR and FFR. This means that both the CFR and FFR are calculated (acquired) from “CT image”. Also in FIG. 20B, a drawing line is drawn from inside the plot, and “CT” is shown. However, in practice, “CT” is shown inside the plot.

In the embodiments described above, a case where an operator specifies all the regions on the image has been explained. However, the embodiments are not limited thereto, and for example, even when a region on the image is indirectly specified, and when a region on the image is specified by using dominant region information, display information is generated and displayed by performing the same processing as the processing described above.

In the embodiments described above, a case where the heart is an object to be diagnosed and treated and the CFR, FFR, and QCA are used as the indexes has been explained. However, the embodiments are not limited thereto, and for example, another internal organ can be the object to be diagnosed and treated. In this case, flow reserve, fractional flow reserve, stenosis ratio, and the like of the internal organ to be diagnosed and treated are used as the indexes.

In the embodiments described above, a case where the medical information processing apparatus 100 generates and displays display information has been explained. However, the embodiments are not limited thereto, and for example, the medical image diagnostic apparatus 200 can generate and display the display information. That is, for example, the medical information processing apparatus 100 can be incorporated in the medical image diagnostic apparatus 200. In other words, the control unit of the medical image diagnostic apparatus 200 includes the data acquisition unit 151, the calculation unit 152, the generation unit 153, and the display control unit 154 described above, to perform the processing described above.

### Another Embodiment

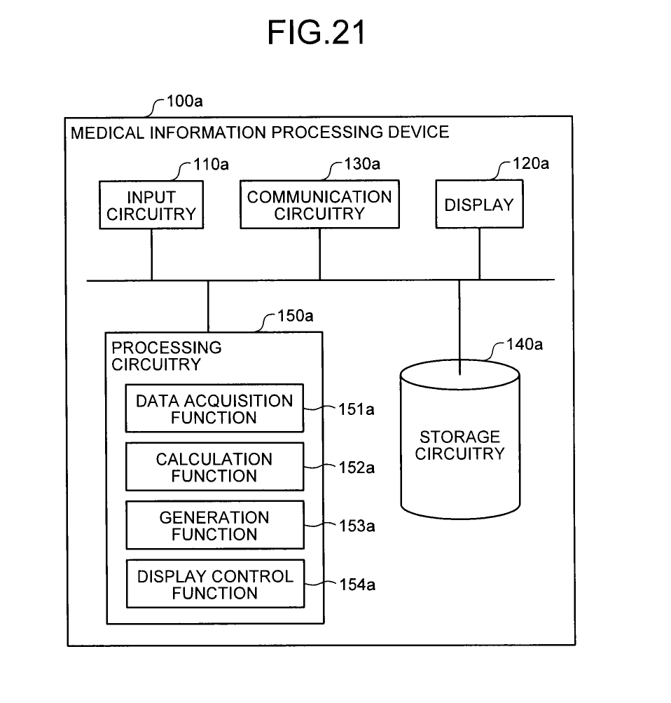

Another embodiment of the medical information processing apparatus described above will be described with reference to FIG. 21. FIG. 21 is a diagram illustrating an example of the configuration of a medical information processing apparatus 100a according to another embodiment. As illustrated in FIG. 21, the medical information processing apparatus 100a according to another embodiment includes an input circuitry 110a, a display 120a, a communication circuitry 130a, a storage circuitry 140a, and a processing circuitry 150a. As illustrated in FIG. 21, each circuitry is connected in each other and to transmit and receive various signals to each other.

The input circuitry 110a corresponds to the input unit 110 illustrated in FIG. 5. The display 120a corresponds to the display unit 120 illustrated in FIG. 5. The communication circuitry 130a corresponds to the communication unit 130 illustrated in FIG. 5. The storage circuitry 140a corresponds to the storage unit 140 illustrated in FIG. 5. The processing circuitry 150a corresponds to the control unit 150 illustrated in FIG. 5.

In the present embodiment, the respective processing functions performed by the communication unit 130 and the control unit 150 illustrated in FIG. 5 are stored in the storage circuitry 140a, in the form of a computer-executable program. Each of the communication circuitry 130a and the processing circuitry 150a is a processor that loads programs from the storage circuitry 140a, and executes the programs so as to implement the respective functions corresponding to the programs. In other words, each circuitry that has loaded the programs has the functions corresponding to the programs loaded.

The term “processor” used in the above description means, for example, a central preprocess unit (CPU) and a graphics processing unit (GPU), or a circuit such as an application specific integrated circuit (ASIC), a programmable logic device (for example, a simple programmable logic device (SPLD)), a complex programmable logic device (CPLD), and a field programmable gate array (FPGA). The processor implements a function by loading and executing a program stored in a storage circuit. Instead of being stored in a storage circuit, the program may be built directly in a circuit of the processor. In this case, the processor implements a function by loading and executing the program built in the circuit. The processors in the present embodiment are not limited to a case in which each of the processors is configured as a single circuit. A plurality of separate circuits may be combined as one processor that implements the respective functions.

The storage circuitry 140a, for example, stores therein computer programs corresponding to a data acquisition function 151a, a calculation function 152a, a generation function 153a, and a display control function 154a illustrated in FIG. 21. The processing circuitry 150a reads the program corresponding to the data acquisition function 151a from the storage circuitry 140a and executes the program, thereby performing processing similar to the data acquisition unit 151. The processing circuitry 150a reads the program corresponding to the calculation function 152a from the storage circuitry 140a and executes the program, thereby performing processing similar to the calculation unit 152. The processing circuitry 150a reads the program corresponding to the generation function 153a from the storage circuitry 140a and executes the program, thereby performing processing similar to the generation unit 153. The processing circuitry 150a reads the program corresponding to the display control function 154a from the storage circuitry 140a and executes the program, thereby performing processing similar to the display control unit 154. The storage circuitry 140a, for example, also stores therein computer programs corresponding to a processing function to control the entire of the X-ray diagnostic apparatus 100a. The processing circuitry 150a reads the program corresponding to the processing function from the storage circuitry 140a and executes the program, thereby performing processing similar to the control unit 150.

The example illustrated in FIG. 21 describes a case of implementing the data acquisition function 151a, the calculation function 152a, the generation function 153a, and the display control function 154a by causing one processing circuitry 150a to execute the respective programs. However, embodiments are not so limited, and for example, a plurality of processing circuits may implement the data acquisition function 151a, the calculation function 152a, the generation function 153a, and the display control function 154a. For example, one or more functions among the data acquisition function 151a, the calculation function 152a, the generation function 153a, and the display control function 154a may be separately implemented in exclusive, independent program execution circuits.

Some of the circuitry illustrated in FIG. 21 may be implemented as one processing circuit. For example, one program execution circuit may implement the communication function implemented by the communication circuitry 130a, the data acquisition function 151a, the calculation function 152a, the generation function 153a, the display control function 154a, and processing function implemented by the processing circuitry 150a.

The input circuitry 110a is implemented by a trackball, a switch button, a mouse, a keyboard, or the like for performing the setting of a ROI (region of interest) or the like. The input circuitry 110a is connected to the processing circuitry 150a, converts input operation received from an operator into an electric signal, and outputs the electric signal to the processing circuitry 150a.

Step S101 and step S102 in FIG. 15 is a step implemented by causing the processing circuitry 150a to read the program corresponding to the data acquisition function 151a from the storage circuitry 140a and to execute the program. Step S103 and step S106 in FIG. 15 is a step implemented by causing the processing circuitry 150a to read the program corresponding to the calculation function 152a from the storage circuitry 140a and to execute the program. Step S104 in FIG. 15 is a step implemented by causing the processing circuitry 150a to read the program corresponding to the generation function 153a from the storage circuitry 140a and to execute the program. Step S105 in FIG. 15 is a step implemented by causing the processing circuitry 150a to read the program corresponding to the display control function 154a from the storage circuitry 140a and to execute the program. The above-described processing circuitry 21a is an example of a processing circuitry in the claims.

According to the medical information processing apparatus according to at least one of the embodiments described above, complex usage of a plurality of indexes can be simplified.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing apparatus comprising:
processing circuitry configured to
generate display information indicating a state of a first region in a tissue of a subject and a state of a second region in a feeding vessel of the first region, depending on flow reserve of the first region and fractional flow reserve of the second region, and
execute control such that the display information generated by the generation unit is shown by a display.

2. The medical information processing apparatus according to claim 1, wherein the processing circuitry is configured to generate, as the display information, information in which a flow reserve value of the first region and a fractional flow reserve value of the second region are shown on a graph in which the flow reserve and the fractional flow reserve are set respectively on a first axis and a second axis.

3. The medical information processing apparatus according to claim 2, wherein the processing circuitry is configured to generate, as the display information, information in which the flow reserve value of the first region, the fractional flow reserve value of the second region, and a value of a stenosis ratio of a stenosis included in the second region are shown on a graph in which the stenosis ratio of the stenosis included in the second region is set on a third axis, in addition to the flow reserve and the fractional flow reserve.

4. The medical information processing apparatus according to claim 2, further comprising input circuitry that receives a value change instruction for at least one of the flow reserve value, the fractional flow reserve value, and the value of the stenosis ratio of the stenosis included in the second region, wherein
the processing circuitry is configured to
regenerate display information indicating a value corresponding to the change instruction received by the input circuitry, and
execute control such that the display information regenerated is shown by the display.

5. The medical information processing apparatus according to claim 2, wherein the processing circuitry is configured to generate, as the display information, information respectively showing values acquired by a plurality of different apparatus on the graph, for at least one of the flow reserve value, the fractional flow reserve value, and the value of the stenosis ratio of the stenosis included in the second region.

6. The medical information processing apparatus according to claim 2, wherein the processing circuitry is configured to generate display information in which the graph is divided to regions for each treatment content decided based on a threshold set to each index set on each axis.

7. The medical information processing apparatus according to claim 6, further comprising input circuitry that receives a change instruction of the threshold set to each index set on each axis, wherein
the processing circuitry is configured to generate display information in which a threshold corresponding to the change instruction received by the input circuitry is set to each of the axes, and the graph is divided to regions for each treatment content based on the set threshold.

8. The medical information processing apparatus according to claim 1, wherein the processing circuitry is configured to generate, as the display information, a composite image in which a first image obtained by color mapping a tissue image of the subject in a color corresponding to the flow reserve value of the first region, and a second image obtained by color mapping an image of the feeding vessel in a color corresponding to the fractional flow reserve value of the second region are synthesized.

9. A medical image diagnostic apparatus comprising:
processing circuitry configured to
generate display information indicating a state of a first region in a tissue of a subject and a state of a second region in a feeding vessel of the first region, depending on flow reserve of the first region and fractional flow reserve of the second region, and
execute control such that the display information generated by the generation unit is shown by a display.

10. A medical information processing method executed by a medical information processing apparatus that processes medical information, the method comprising:
generating display information indicating a state of a first region in a tissue of a subject and a state of a second region in a feeding vessel of the first region, depending on flow reserve of the first region and fractional flow reserve of the second region; and
executing control such that the generated display information is shown by a display.
